(19) United States
(12) Patent Application Publication
(10) Pub. No.: US 2008/0026429 A1
(43) Pub. Date: Jan. 31, 2008

(54) Solution pre-treatment

(75) Inventors: Hugh William Morgan, Hamilton (NZ); Andreas Rueckert, Hamilton (NZ)

Correspondence Address: GREER, BURNS & CRAIN, 300 S WACKER DR, 25TH FLOOR, CHICAGO, IL, 60606, US

(73) Assignee: WaikatoLink Limited

(21) Appl. No.: 11/717,509

(22) Filed: Mar. 13, 2007

Related U.S. Application Data
(60) Provisional application No. 60/781,641, filed on Mar. 13, 2006.

(51) Int. Cl. C12P 19/34; C07C 1/00

(52) U.S. Cl. 435/91.2; 204/157.15

(57) ABSTRACT

A method of preparing a solution for use in DNA amplification reactions, including the steps of, mixing a binding agent with a base solution; subjecting the mixture (of a binding agent and base solution) to a stimulus which activates the binding agent, and upon activation, the binding agent binding to any DNA in the base solution so that it is unreactive and is not co-amplified during the DNA amplification reaction.

### TECHNICAL FIELD

This invention relates to a solution pre-treatment.

More specifically this invention relates to the pre-treatment of protein solutions.

Preferably the protein solutions are for use in deoxyribonucleic acid (DNA) amplification reactions, and the pre-treatment ensures that any contaminating DNA present in the protein solution cannot take part in the DNA amplification reaction.

### BACKGROUND ART

Amplification of nucleic acids, especially DNA has become a widely used tool in DNA analysis work. This is especially so in cases where only minute amounts of DNA are present.

Currently, the most commonly used method of DNA amplification is the polymerase chain reaction (PCR).

PCR is a method of amplifying a DNA base sequence using a heat-stable DNA polymerase and two approximately 20-base primers, one complementary to the (+)-strand at one end of the sequence to be amplified and the other complementary to the (−)-strand at the other end of the sequence to be amplified.

Because the newly synthesized DNA strands can subsequently serve as additional templates for the same primer sequences, successive rounds of primer annealing, strand elongation, and dissociation produce rapid and highly specific amplification of the desired sequence.

PCR also can be used to detect the existence of a defined sequence in a DNA sample, if relevant primers can be developed.

The contamination of DNA polymerase and other protein PCR reagents with bacterial (or other) DNA is a major problem for PCR methodology. This problem has previously been addressed by several authors (Böttger, 1990; Rand and Houck, 1990; Schmitd et al., 1991; Hughes et al., 1994; Maiwald et al., 1994; Hilali et al., 1997).

The ability of PCR to amplify minute amounts of DNA is one of its greatest strengths. However, this ability also means that any traces of contaminating DNA are also co-amplified. This amplification of contaminating DNA can produce misleading, ambiguous or incorrect results (Wilson et al., 1990).

In particular, the concept of targeting the highly conserved and evolutionarily homologous eubacterial ribosomal 16S rRNA genes with universal primers for phylogenetic, evolutionary and diagnostic studies is often compromised by the generation of false positives during PCR (Hilali et al., 1997; Corless et al., 2000; Mohammadi et at., 2003).

Eubacterial DNA is ubiquitous in the environment and contaminating DNA can also be introduced into the PCR mix at practically any stage of processing by laboratory personnel (Kitchin et al., 1990; Millar et al., 2002).

Other sources of DNA contamination include: aerosols (Saksena et al., 1991), consumable PCR reagents, plastic ware (Millar et al., 2002) or commercial PCR primers (Goto et al, 2005).

In particular, commercially available DNA polymerases have been repeatedly reported to be one of the most likely sources of exogenous bacterial DNA contamination (Rand and Houck, 1990; Böttger, 1990; Schmidt et al., 1991; Hilali et. al., 1997; Newsome et al., 2004).

Schmidt et al., (1991) regard micro-organisms used for the production of DNA polymerases and enzyme purification equipment, such as chromatography columns, as the most likely source for introducing exogenous DNA into DNA polymerases.

In contrast, two other studies found no evidence that the exogenous bacterial DNA in Taq-polymerase originates from the producing organisms such as Escherichia coli or Thermus aquaticus (Rand and Houck, 1990; Maiwald et al, 1994) and the contaminating DNA was shown to be closely related to Pseudomonas fluorescens, Pseudomonas aeruginosa, Alcaligenes faecalis and Azotobacter vinelandii (Maiwald et al, 1994).

In addition, Schmidt et al. (1991) have also detected contaminating DNA originating from eukaryotes in various batches of Taq-polymerase preparations.

Regardless of source however, these studies serve to emphasize that exogenous DNA is ubiquitously present in commercially available Taq-polymerase preparations.

Strategies to eliminate this contaminating DNA include treatment of the PCR master mix preparation with UV light (Ou et al., 1991; Sarkar and Sommer, 1991; Frothingham et al, 1992; Sarkar and Sommer, 1993; Goldenberger and Altwegg, 1995), DNase I (Rochelle et al, 1992; Hilali et al, 1997), gamma-irradiation (Deragon et al., 1990), restriction enzymes (DeFililppes, 1991; Sharma et al, 1992; Mohammadi et al, 2003) or 8-methoxypsoralen combined with UV irradiation (Jinno et al, 1990; Meier et al, 1993; Hughes et al, 1994; Fahle et al., 1999). However, the latter methodology could not successfully be reproduced in the studies of Schmidt et al, (1991) and Corless et al, (2000), respectively.

These methods however all have significant disadvantages.

For example the use of UV irradiation (256 nm) to eliminate exogenous DNA is problematic as Taq polymerase has been shown to be extremely sensitive to this treatment (Ou et al, 1991) with reductions of amplification efficiencies by more than 105-fold (Sarkar and Sommer, 1990).

Similarly, many primers (depending on their sequence) are also sensitive to UV irradiation, whereas deoxynucleotide triphosphates are essentially unaffected by the UV treatment but may reduce the efficiency of decontamination (Frothingham et al, 1992).

The most effective procedure to date for the reduction of unwanted DNA has been the pre-treatment of PCR reagents with DNase I and/or restriction enzymes (Sharma et al, 1992; Hilali et al, 1997; Carroll et al, 1999; Heininger et al, 2003; Mohammadi et al, 2003).

However, the use of DNase I requires a thorough heat denaturing step to eliminate any residual enzyme activity before the template DNA can be added to the reaction.

Hilali et al, (1997) reported that high temperatures and extended periods of incubation (95° C. for 50 minutes or 100° C. for 20 minutes) were essential for the efficient inactivation of DNase I. Such treatments are not compatible with the thermo-stability of most Taq-polymerases. If not completely inactivated, then residual DNase I could compromise the template DNA. To circumvent the reduction in the PCR efficiency the employment of the highly thermo-stable Deepvent Exo-® polymerase has been recommended.

The use of restriction enzymes can also be problematic as the enzymes themselves may be a source of exogenous eubacterial DNA (Jinno et al, 1990).

There has been some work in the area of capturing DNA after amplification as discussed in NZ 514707. However, this composition and method requires the use of binding enhancers and a label which is used for subsequent analysis and detection of amplicons.

It would therefore be beneficial if a method of quick and easy pre-treatment of protein solutions where available which could lead to the decontamination or prevent any contaminating DNA from taking part in any future PCR reactions or other DNA amplification method.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a method of preparing a solution for use in DNA amplification reactions, including the steps of:
a) mixing a binding agent with a base solution,
b) subjecting the mixture from a) to a stimulus which activates the binding agent, causing, the binding agent to bind to any DNA in the base solution so that it is unreactive, and is not co-amplified during the DNA amplification reaction.

According to a second aspect of the present invention there is provided a solution for use in DNA amplification reactions wherein the solution is produced via the method substantially as described above.

According to another aspect of the present invention there is provided a method of DNA amplification, including the steps of:
a) preparing a solution using the method substantially as described above, and
b) using the solution from a) in a DNA amplification reaction.

According to another aspect of the present invention there is provide a method of preparing a solution for use in DNA amplification reactions, including the steps of:
a) determining the amount of contaminating exogenous DNA in a base solution,
b) determining the concentration of a binding agent required to bind all the contaminating DNA
c) mixing the required concentration of binding agent with a base solution,
d) subjecting the mixture from (c) to a stimulus which activates the binding agent, and
e) upon activation, the binding agent binding to any DNA in the base solution so that it is unreactive and is not co-amplified during the DNA amplification reaction.

In a preferred embodiment the base solution may be a protein solution, and shall be referred to as such herein.

One skilled in the art however, would readily realise that the same procedure to bind contaminating DNA could be used in any other solutions which are required to be nucleic acid free, such as magnesium chloride solutions for use in PCR or other amplification reactions.

It is envisaged that nearly all methodologies using an enzyme to amplify DNA are likely to contain contaminating DNA, and this method would allow removal of the contaminating DNA without affecting enzyme activity. For example, ligase chain reaction, nucleic acid sequence-based amplification, strand displacement amplification and transcription-mediated amplification.

Throughout this specification the term ‘base solution’ should be taken as meaning any protein or other solution which has been prepared or is commercially available for use in DNA amplification reactions, but which may still include contaminating DNA.

In one preferred embodiment the protein solution may be a solution of DNA polymerase, used for the extension of nucleic acid strands in PCR reactions. However this should not be seen as limiting as the present invention may also be used for other protein solutions for DNA amplification or detection, for example restriction enzymes.

In one particularly preferred embodiment the protein solution may be a solution of Taq polymerase, a heat-stable DNA polymerase isolated from the bacterium Thermus aquaticus.

In an alternative embodiment the base solution may be a PCR master mix, containing all the required components to carry out a PCR reaction except the template DNA to be amplified.

In one embodiment the base solution may be an RNA preparation. RNA analysis often includes the steps of: extracting RNA from a cell, making DNA copies (cDNA) of the RNA (eg mRNA, rRNA) molecules using a reverse transcriptase and then amplifying the cDNA by PCR, or other amplification techniques.

For accurate results it is important that the RNA extract is not contaminated with cellular DNA.

Therefore, in one embodiment the base solution may be an RNA extract prior to cDNA production.

Protein solutions obtained from any source are likely to contain contaminating DNA.

For example, the inventors have investigated three of the most commonly used commercially available Taq polymerase preparations. All three contained roughly the same level of contaminating DNA.

Commercially available preparations of Taq polymerase must be screened to be below an “acceptable” level of DNA contamination, however, this does not mean they are contamination free.

It is commonly accepted by researchers that false positives, caused by amplification when no template DNA is added will occur on extended PCR runs i.e. above 36 cycles. Many researchers will therefore limit the PCR run length to achieve positive results below the 36-40 cycle number. To do this it is necessary to ensure sufficient template DNA is added to overwhelm the effect of small amounts of contaminating DNA.

The co-amplification of contaminating DNA is most likely to occur when using generic or universal primers. Universal primers are those that, for example will bind to DNA from any Eubacteria species.

The current applicants found that during studies targeting the 16S rRNA gene with PCR severe problems with the generation of false positives in the negative control samples with no added template DNA were encountered.

After extensive trial experiments using new batches of PCR buffer solutions, magnesium chloride, deoxynucleotide triphosphates, primers, millipore water preparation, plastic ware and aerosol-resistant filter tips the conclusion was that the source of contamination was the AmpliTaq® DNA polymerase itself.

As the contaminating DNA is only noticeable, or a problem when it is amplified to detectable levels, the present invention is targeted at use for solutions which are used in DNA amplification reactions.

Throughout this specification, amplification reactions shall be referred to as PCR, as this is the most commonly used amplification technique, however this should not be seen as limiting as one skilled in the art would realise that the current invention may be utilised as a pre-treatment of solutions for any amplification technique.

Throughout this specification the generic meaning of PCR shall be taken, that is: using a heat-stable polymerase and two approximately 20-base primers, one complementary to the (+)-strand at one end of the sequence to be amplified and the other complementary to the (−)-strand at the other end. Repetitive cycles of primer annealing, strand elongation and dissociation produce rapid and highly specific amplification of the desired sequence.

The present invention provides a quick and easy method of binding contaminating DNA and preventing co-amplification of same. The current method of preventing any contaminating DNA from participating in DNA amplification reactions could either be undertaken by a commercial supplier, thereby being able to provide guaranteed DNA free polymerase or master mixes, alternatively the current method could be undertaken by individual research institutions or researchers as and when required.

Throughout this specification the term DNA should be taken as having its generic meaning, including any compound made up of purine or pyrimidine base nucleotides, each with a deoxyribose sugar, and phosphate group.

In order to prevent further contamination of the protein solution it is preferable that the binding agent will prevent contaminating DNA from taking part in amplification reactions requiring the least possible number of opening, adding or removals from the reaction tube.

In a preferred embodiment the binding agent may be one which has two different forms, one being an inactive form wherein it will not react or bind to DNA, and one being an active form wherein it will bind to DNA.

It must be possible for the binding agent to be able to go between the inactive and active form (and vice versa) easily and quickly.

This means that a sufficient quantity of the binding agent to bind to the contaminating DNA can be added to the solution, and will only bind with the contaminating DNA when in its activated state.

This means that once some of the binding agent has bound with all contaminating DNA the remaining agent can be reverted to the inactive form and will not bind to any further DNA.

This prevents the binding agent/contaminating DNA combination from interfering in any further reaction, or during DNA amplification once the target DNA has been added.

It is important that the activation and deactivation of the binding agent is easily undertaken and controllable. This ensures that binding of the agent to any contaminating DNA will only take place prior to the addition of template (target) DNA and initiation of the DNA amplification reaction.

In a preferred embodiment the binding agent will only be in an active form in the presence of a stimulus.

This is important as it means that once the binding agent is removed from the activating stimulus it will not be able to bind to DNA, especially template DNA which may then be added to the protein solution, or protein solution and other components required for amplification.

Another important feature of being able to control when the binding agent is activated is that it means that the binding agent and bound DNA do not have to be removed from the protein solution prior to DNA amplification.

This is advantageous in that it reduces the possibility for further contamination by opening the container/reaction tube.

The binding agent does not break down or destroy the contaminating DNA, this is also advantageous as such fragments or portions may themselves interfere in the amplification reaction.

Another advantage is that there is no possibility of any other solution constituents, such as enzymes being affected by the binding agent.

In a preferred embodiment the binding agent may be activated by an external stimulus such as UV or ordinary light. However, this should not be seen as limiting as the stimulus will depend on the binding agent selected, and stimuli which may result in the binding agent being transferred from an inactive to an active form (or vice versa).

In one preferred embodiment the binding agent may be an intercalating agent.

Throughout this specification the term intercalating agent should be taken to include any agent that is capable of intercalating with DNA, that is to be reversibly included between two adjacent base pairs. Some well studied DNA intercalators include ethidium, proflavin and thalidomide.

In one particular preferred embodiment the binding agent may be ethidium monoazide (EMA).

In this case, the method is based on the complexation of the contaminating DNA with the intercalating agent EMA. EMA is a photoreactive analogue of ethidium bromide, which in the dark, interacts with DNA in a similar manner as its parent compound (Bolton and Kearns, 1978; Garland et al, 1980).

In a preferred embodiment the amount of binding agent added may be just sufficient to bind contaminating DNA.

In the case when EMA is the binding agent, EMA may preferably be used at a concentration of between 1 and 5 μg ml-1.

In a particularly preferred embodiment the EMA concentration in the order of 3 μg ml-1 may be used.

An EMA concentration of 3 μg ml-1 may be sufficient to completely eliminate contaminating DNA from the master mix preparations, without affecting the amplification of template DNA concentrations as low as 10 fg

Optimally, EMA would be added to a minimal titre, just sufficient to bind all exogenous DNA, while minimizing the formation of free nitrene in solution associated with the formation of hydroxylamine.

At EMA concentrations greater than 5 μg ml⁻¹ inhibition of PCR was evident for those reactions containing less than 100 pg of template DNA.

The amount of contaminating DNA in commercially available PCR reagents will vary. Therefore every batch of DNA polymerase or PCR master mix will require optimization of the EMA titer to be undertaken to ensure maximal removal of contaminating DNA while maintaining PCR sensitivity.

EMA is an analogue of ethidium bromide in which the amino group in the 8-position has been replaced with an azido group. EMA intercalates with double stranded DNA in the dark in the same manner as its parent compound but can additionally covalently bind DNA after photo-activation with visible light (Bolton and Kearns, 1978; Garland et. al., 1980).

Accordingly, DNA that has chemically reacted with the azide, is unable to be amplified by PCR.

Thus, the addition of a binding agent, such as EMA can be used to bind to and prevent exogenous DNA from being co-amplified in Taq polymerase and PCR reagents, by adding this component to a master mix which contains all amplification components, apart from the template DNA.

On photolysis, the azido-group in the 8-position of EMA is photo-chemically activated using long wavelength light greater than substantially 400 nm (Bolton and Kearns, 1978) to covalently crosslink with nucleic acid at the site of binding via a nitrene radical (Hardwick et al., 1984; Cantrell et al., 1978). Residual unbound nitrene in the solution is converted to hydroxylamine (Graves et al., 1981), which reacts with aldehyde and/or ketone components of the solution—preferentially with cytosine—to form oximes (Freese et al., 1961). The covalently cross-linked DNA is unable to participate in PCR amplification and therefore its interference is removed (Nogva et al., 2003).

Throughout this specification the term unreactive in relation to contaminating DNA should be taken as meaning unable to be amplified by DNA amplification techniques.

This means that although the bound contaminating DNA remains in the solution, the DNA level remains at its original low level which is not detectable by usual detection methods used after DNA amplification.

Throughout this specification the term Master mix should be taken as meaning a mixture of some, or all of the required components for a PCR reaction, but excluding the template DNA to be amplified.

For example PCR master mix may contain: 1X Taq Master Mix:

10 mM Tris-HCl

50 mM KCl

1.5 mM MgCl₂

0.2 mM dNTPs

0.5 mM DTT

5% glycerol

0.8% NP-40

0.05 mM Tween-20

25 U/ml Taq DNA Polymerase

Components for PCR/amplification reactions can vary quite markedly depending on the researcher and the aim.

Some additives are optional eg DTT, glycerol, Tween, others are always present but the concentration used can vary, for example primers and MgCl.

The composition of the master mix is not so important as most of the contaminating DNA is introduced with the Taq polymerase and this is nearly always used at between 0.5-2.0 units per reaction (with 1 unit per reaction considered a good average).

Throughout this specification the term template DNA should be taken as meaning target DNA which is amplified during a PCR reaction.

In the present invention, we present a novel and rapid methodology to remove any exogenous double-stranded DNA from a PCR master mix in less than 10 minutes.

Advantages of the current invention include the following:
It provides a quick and easy method for preventing contaminating DNA in protein or other solutions from being co-amplified in DNA amplification reactions such as PCR,
Capturing DNA before amplification means minimal template DNA for amplification is required and PCR runs to excess of 36 cycles can be undertaken with confidence that false positives will not be produced,
It makes use of readily available, over the counter compounds, thus reducing the cost and preparation time,
It will allow for the binding of DNA in the presence of enzymes or other protein or chemical constituents, without a detrimental effect on same,
The binding agent does not lead to the break down of contaminating DNA thereby preventing fragments of same interfering in DNA amplification reactions,
The binding agent, with bound contaminating DNA does not have to be removed from the solution, thus preventing further possible contamination by limiting the number of times the container/reaction tube needs to be opened.
The control of when the binding agent is active, by using a stimulus such as UV light prevents the binding agent, or binding agent bound to DNA from interfering in any subsequent amplification reaction.
The binding agent does not require preparation, other than adjustment to the correct concentration before the method is undertaken,
the binding agent, or binding agent bound to contaminating DNA does not inhibit Taq polymerase,
Only a low level of the binding agent is required to be added to the base solution or reaction mixture.

### BRIEF DESCRIPTION OF DRAWINGS

Further aspects of the present invention will become apparent from the following description which is given by way of example only and with reference to the accompanying drawings in which:

FIG. 1 illustrates PCR amplification product with decreasing template DNA concentration in the presence of 0.1 μg ml⁻¹ EMA, and pixel density profile of product concentration,

FIG. 2 illustrates pixel density profile for 1 μg ml⁻¹;

FIG. 3 illustrates pixel density profile for 3 μg ml⁻¹;

FIG. 4 illustrates pixel density profile for 5 μg ml⁻¹,

FIG. 5 illustrates pixel density profile for 10 μg ml⁻¹.

FIG. 6 illustrates pixel density profile using 10 μg ml⁻¹ EMA and 200 μg bovine DNA!

FIG. 7 illustrates pixel density profile for 3 μg ml⁻¹ EMA on JumpStart™ Taq DNA polymerase, and

FIG. 8 illustrates pixel density profile for 3 μg ml⁻¹ EMA on Platinum® Taq DNA Polymerase.

### BEST MODES FOR CARRYING OUT THE INVENTION

The results presented demonstrate that EMA is capable of completely and rapidly eliminating any amplifiable exogenous DNA from a PCR master mix preparation within 10 minutes.

The amount of EMA required to decontaminate a PCR master mix preparation depends on the amount of contaminating DNA present.

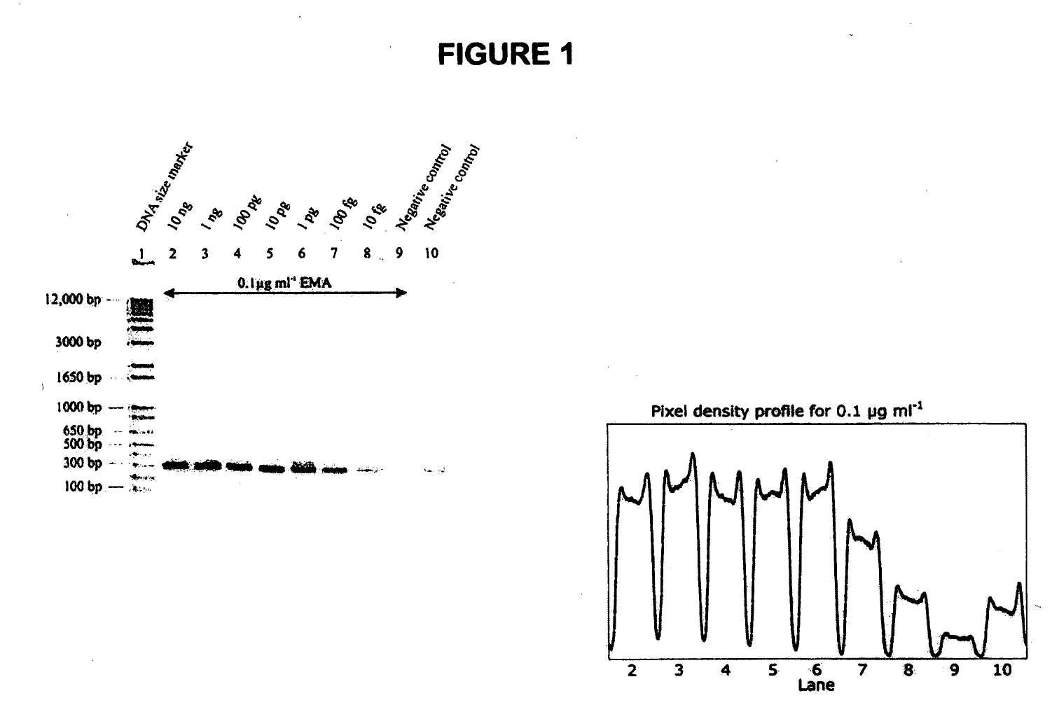

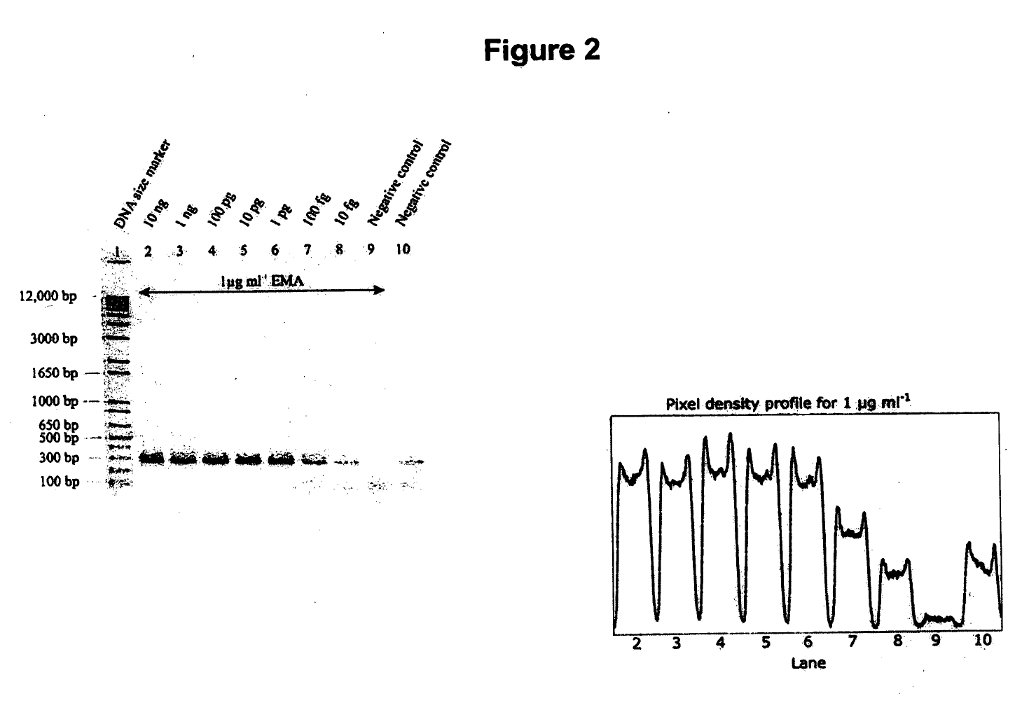

As shown in FIGS. 1 and 2, azide titres of 0.1 and 1 μg ml⁻¹ EMA were unable to completely eliminate all contaminating DNA from the PCR preparation, although, quantitatively more exogenous DNA was removed applying 1 μg ml⁻¹ (pixel density profile of FIG. 1 and 2, lanes 9).

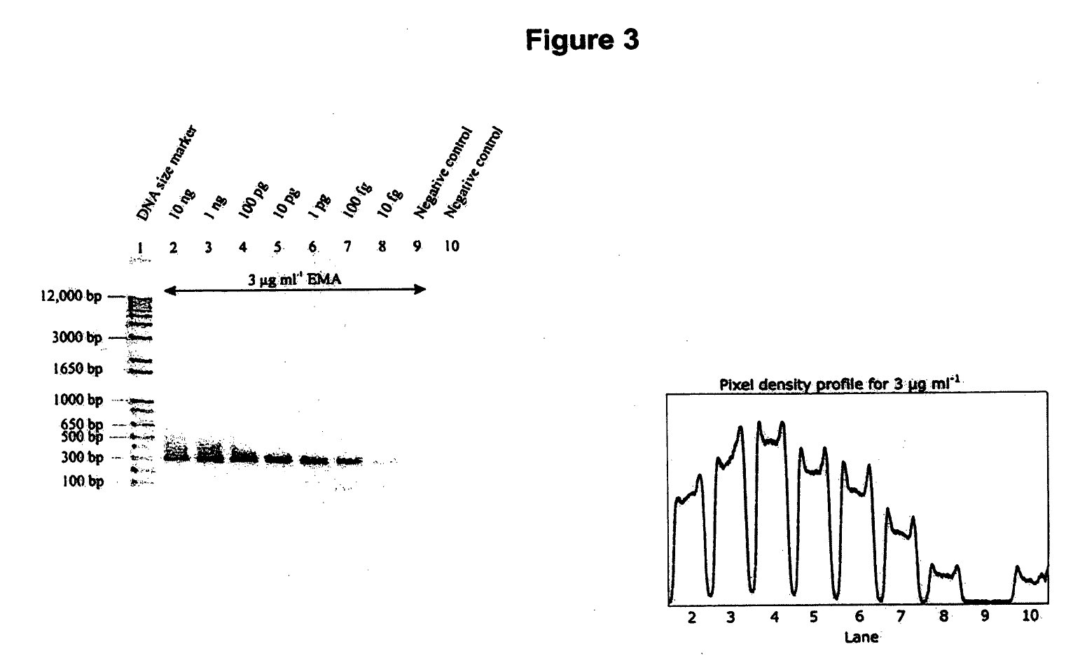

An EMA concentration of 3 μg ml⁻¹ was required to completely eliminate the contaminating DNA from the master mix preparations. This concentration did not affect the amplification of template DNA concentrations as low as 10 fg (FIG. 3, lane 8).

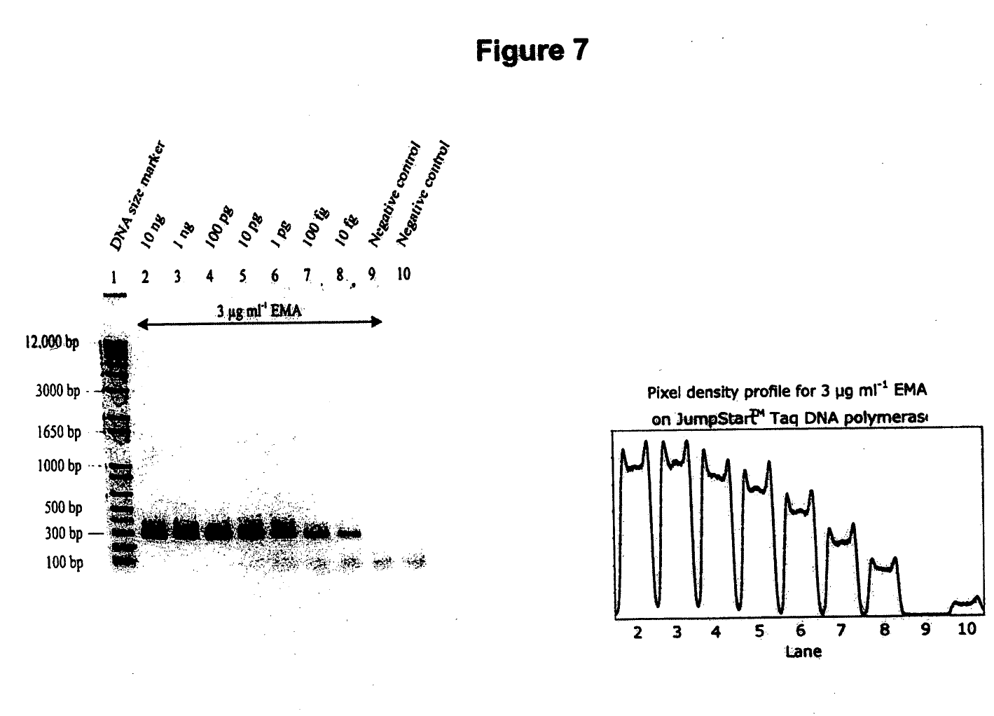

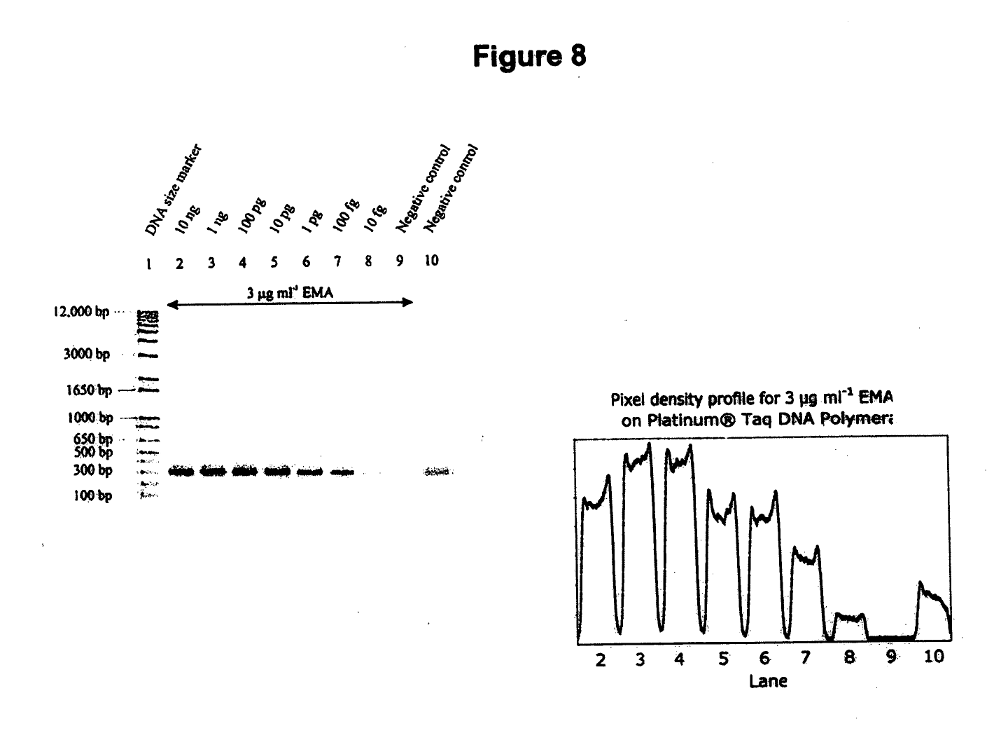

The same concentration of EMA (3 μg ml⁻¹) was required to completely eliminate contaminating DNA in the two other Taq-polymerases tested: JumpStart Taq-polymerase and Platinum Taq-polymerase (FIGS. 7 and 8, lanes 9). Again template DNA concentrations of 10 fg DNA were still amplified without any adverse effect (FIGS. 7 and 8, lanes 8).

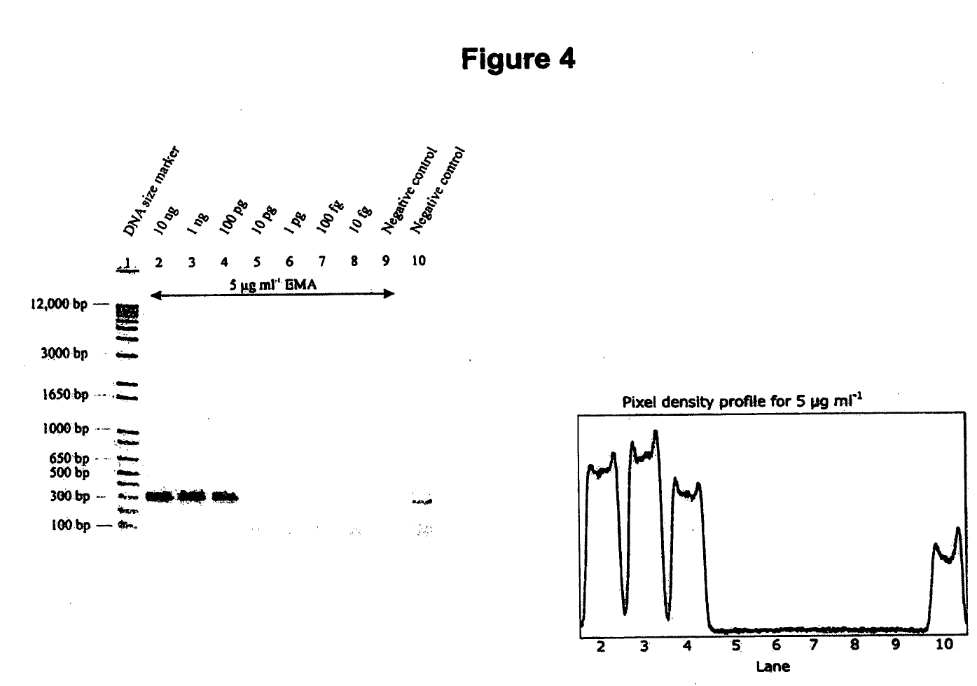

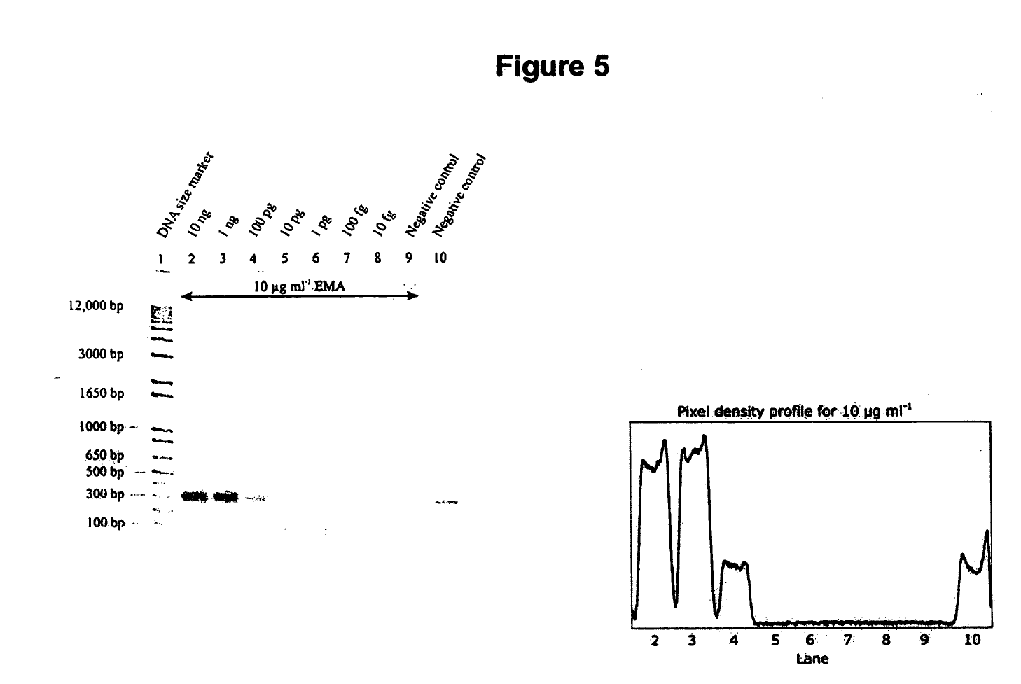

At EMA concentrations greater than 5 μg ml⁻¹ inhibition of PCR was evident for those reactions containing less than 100 pg of 17 template DNA (FIGS. 4 and 5, lanes 5 to 8).

Interestingly, the addition of 200 pg μl⁻¹ of digested bovine DNA to an identically prepared master mix restored PCR performance, indicating that amplification failure for template concentrations below 100 pg was not due to the inhibition of the Taq-polymerase but due to template removal by unbound EMA.

In contrast, an aliquot of 20 pg ∥l⁻¹ of digested bovine DNA, was not sufficient to remove PCR inhibition (data not shown).

The evaluation of the PCR products after agarose-gel electrophoresis was performed with the free downloadable software ImageJ provided by the National Institute of Health, USA.

This software was used to calculate the pixel value statistics of a selected area in the agarose gel images, from which the line density profile plots were established. This computer controlled measurement of the relative quantities of the amplicons had advantages over direct visual observation, particularly for the negative controls. This method minimized ambiguous results due to misinterpretations or inconsistencies in ethidium bromide staining procedures and gel to gel variations.

Thus, the inventor regards the use of the ImageJ or a similar computer program to monitor the extent of decontamination advisable.

The amount of contaminating DNA in commercially available PCR reagents will vary and every batch of DNA polymerase or PCR master mix will require an optimization of the EMA titer to ensure maximal removal of contaminating DNA while maintaining PCR sensitivity.

Optimally, EMA would be added to a minimal titer - just sufficient to bind all exogenous DNA, while minimizing the formation of free nitrene in solution associated with the formation of hydroxylamine.

Hydroxylamine has been shown to be mutagenic, being able to bind and chemically modify DNA over the concentration range of 250 to 500 mM, with incubation times greater than 30 minutes (Aslanzadeh, 1992; Freese et al., 1961). In addition, hydroxylamine induces DNA damage in the presence of Cu(II), but not in the presence of Mn(III), Mn(II), Fe(III) or Fe(II) (Yamamoto et al., 1993).

Fortunately, the inventor could demonstrate that the optimal azide titre of 7.14 μM (3 μg ml⁻¹) had no adverse affect on the template DNA amplified as shown by sequencing of the D-alanine racemase gene from B. subtilis BS. Bolton and Kearns (1978) reported that the yield for cross-linking of EMA to DNA is constant with a value of 75%, for a low ratio of EMA per nucleic acid. Consequently, the amount of unbound and reactive hydroxylamine in the PCR master mix is effectively 1.78 μM at the optimal EMA titer of 3 μg ml⁻¹.

Even when EMA was added in excess of that required to remove exogenous DNA, with concentrations of 11.9 μM (5 μg ml⁻¹) there was no alteration in the sequence of amplified DNA, although the proportion of reactive hydroxylamine at this concentration is 6.54 μM.

Thus, the current methodology will have no adverse effects on subsequent cloning and sequencing reactions which may be undertaken on the amplicons.

Theoretically, the EMA optimization step could be redundant when the template DNA concentration is above 100 pg, i.e. where a small loss of template DNA due to unbound EMA can be sustained. In these situations an EMA concentration of 4 to 5 μg ml⁻¹ could be recommended.

In conclusion, the current method is able to completely remove any contaminating DNA associated with commercially available DNA polymerases and/or PCR reagents from being co-amplified during PCR after 41 cycles of amplification (i.e. with no added template DNA).

Significantly, the concentration of 3 μg ml⁻¹ EMA was effective in both eliminating contaminating DNA and not interfering with PCR amplification at very low template concentrations. This was validated for PCR master mix preparations containing three different Taq polymerases.

The decontamination protocol presented is rapid and simple to employ, being accomplished within 10 minutes on PCR amplifications.

Thus, the current methodology is well suited as a routine molecular diagnostic tool for the analysis of subtle/delicate PCR samples with extremely low template concentrations where no background amplification is desired, i.e. the analysis of clinical and forensic specimens.

Furthermore, the method can be combined with quantitative real-time PCR applications although it must be borne in mind that EMA is a fluorescent dye with different excitation and emission wavelengths for the bound, unbound and photolysed state (Bolton and Kearns, 1978; Garland et. al., 1980; Graves et al., 1981). Thus, its integration into a quantitative PCR assay would have to be coordinated with the quencher and/or reporter dye.

In addition, it is likely that the current method would be equally effective on any other enzymes used in molecular biology where DNA contamination might be a problem, for example restriction enzymes and proteases.

EXPERIMENTAL

1. Materials and Methods

1.1. Bacterial Strains and Culture Preparation

Anoxybacillus flavithermus strain C and Bacillus subtilis strain BS were isolated from a New Zealand milk powder by Ronimus et al. (2003). The cultures were grown at 55° C. in tryptic soy broth (TSB) supplemented with 0.2% (w/v) soluble potato-starch (Sigma; S2004).

1.2. Template DNA Preparation

DNA was extracted from A. flavithermus and B. subtilis by a modification of the procedure described by Sambrook and Russel (2001). Following growth, 10 ml of cell suspension was harvested and re-suspended in 1 ml of 50 mM Tris HCl (pH 8.0), 100 mM NaCl and 20 mM EDTA.

A total of 2 mg ml⁻¹ lysozyme and 50 μg ml⁻¹ RNase was added and the samples incubated at 37° C. for 45 minutes. SDS and proteinase K were added to 1% (w/v) and 200 μg ml⁻¹, respectively. The samples were then incubated at 55° C. for 1 hour. Subsequently, the samples were extracted with equal volumes of phenol:chloroform (1:1) followed by chloroform and chloroform:isoamyl alcohol (24:1). The DNA was then precipitated by the addition of 1/10 volume of 3 M sodium acetate and 0.6 volume of isopropanol and incubating at −20° C. for 15 minutes. The DNA pellet was washed twice with ice chilled 80% ethanol, air dried and the DNA pellet re-suspended in 500 μl of 1×TE (10 mM Tris-HCl, 1 mM EDTA, pH 8.0).

Deoxyribonucleic acid from calf thymus was purchased from Sigma (D4764) as lyophilized powder and re-suspended in 1×TE. An aliquot of 25 μg of the bovine DNA was partially digested in a total sample volume of 100 μl with 1 Kunitz units of DNase I (Sigma, DN25) and 10 μl of 10×DNase buffer (10 mmol l⁻¹ EDTA, 75 mmol l⁻¹ MgCl2 and 200 mmol l⁻¹ Tris-HCl, pH 7.5) at 37° C. for 10 minutes. Subsequently, 20 mmol h⁻¹ EDTA was added and the sealed sample incubated in a boiling water bath for 10 minutes and the extent of the uncompleted digestion validated by agarose gel electrophoresis. The partially digested bovine DNA sample was used to complex and remove unbound azide from the PCR master mix. All DNA samples were quantified with a NanoDrop (NanoDrop Technologies, Wilmington, Del. 19810, USA) and stored at −20° C.

Furthermore, in all handling of DNA, measures to eliminate laboratory contamination included the incorporation of aerosol-resistant filter tips for pipetting and processing of all solutions and reagents under a sterile laminar flow hood. All water solutions were UV irradiated (256 nm) in clear 1.5 ml Eppendorf tubes for 45 minutes prior to use.

1.3. Oligonucleotide Primers

The forward (5′-AGG MC ACC AGT GGC GM G-3′) and reverse primer (5′-GGA TGT CM GAC CTG GTA AGG-3′) from the study of Rueckert et al., 2005 were used to amplify a small region of the ribosomal 16S rRNA genes at position 711-998 according to the numbering of Brosius et al. (1980). The size of the PCR amplicon was expected to be approximately 300 bp depending on the template DNA present in the sample.

The D-alanine racemase gene from B. subtilis BS was amplified and sequenced using primers derived from the D-alanine racemase gene B. subtilis strain 168 (NCBI accession number M16207). The forward primer and reverse primer were 5′-AGC ACA AM CCT TTT TAC AGA GAT AC-3′ and 5′-TTA ATT GCT TAT ATT TAC CTG CAA TAA AG-3′, respectively.

1.4. Polymerase Chain Reaction

The following three DNA Taq-polymerases were used in this study viz. AmpliTaq® polymerase (Roche, Cat. No. 11647687001), JumpStart™ Taq DNA polymerase (Sigma, D 9307) and Platinum®) Taq DNA Polymerase (Invitrogen, Cat. No. 10966-026). The reactions were set up with the associated magnesium and PCR buffer solution provided by each manufacturer.

All PCR reactions were performed with the Eppendorf Mastercycler (Gradient) in 0.5 ml microfuge tubes containing a final volume of 25 μl. The amplification protocol employed a modification of the protocol described by Rueckert et al. (2005) with the following cycling conditions: 94° C for 150 seconds then 41 cycles of 9420 C. for 15 seconds, 62° C. for 10 seconds and 68° C. for 15 seconds. The amplification reactions contained 4 mM of either MgCl₂ or MgSO₄, 0.2 mM dNTPs, 1×Taq PCR reaction buffer, 1.25 units of appropriate Taq polymerase, 100 nM of forward and reverse primers and EMA in the range of 0.1 and 10 μg ml⁻¹. The amplification reaction contained DNA from A. flavithermus in the decimal dilution range from 10 ng to 10 fg.

Some PCR reactions also contained calf thymus DNA (after treatment with DNase I) of between 200 and 20 pg μl⁻¹.

Two negative control samples were included for each amplification reaction, both of which contained no added template DNA, and one of which additionally did not contain EMA.

The D-alanine racemase from B. subtilis strain BS was amplified in the presence of 0, 3 and 5 μg ml⁻¹ of EMA. The amplification reaction and sequencing reaction containing EMA were performed in duplicate. The PCR reactions contained 4 mM of MgCl₂, 0.2 mM dNTPs, 1×AmpliTaq® PCR reaction buffer, 1.25 units of AmpliTaq® polymerase, 600 nM of forward and reverse primer and 1 ng of genomic DNA of B. subtilis BS. The PCR reaction was cycled at 94° C. for 150 seconds then 41 cycles of 94° C. for 20 seconds, 47° C. for 20 seconds and 72° C. for 90 seconds. A final elongation step of 10 minutes at 72° C. completed the amplification.

1.5. Sequencing of the D-alanine Racemase

Following PCR, the amplification reactions were electrophoresed through a 1.5% (w/v) agarose gel (SeaKem, LE Agarose) (section 2.8). The gels were then briefly viewed on a UV transilluminator while the D-alanine racemase amplification products were excised with a sterile razor blade. The excised bands were wrapped between sheets of parafilm and frozen at −20° C.

Subsequently, a firm and constant pressure was applied to the wrapped gels and the exudates collected into 1.5 ml Eppendorf tubes. These DNA samples were extracted with phenol:chloroform, chloroform and chloroform:isoamyl alcohol, then further precipitated with sodium acetate/isopropanol and quantified as described.

DNA sequencing was carried out for each sample in the forward and reverse priming direction by the Waikato DNA Sequencing Facility on a MegaBACE capillary DNA Analysis System.

1. 6. Ethidium Monoazide Treatment of PCR Master Mix

Five milligrams of solid ethidium monoazide bromide (EMA) were purchased from Biotium, Inc. (USA) and according to the manufacturer's recommendation dissolved in N,N-dimethylformamide in the dark. Aliquots of 50 μl of the 10 mg ml⁻¹ EMA stock solution were stored at −20° C. in light-impermeable micro-tubes.

EMA was added in concentrations of 0.1, 1, 3, 5 and 10 μg ml⁻¹ (diluted in sterile Millipore water) to the PCR master mix preparation to complex contaminating DNA prior to photolysis and the addition of template DNA of A. flavithermus.

Crucially, the addition of EMA occurred in a dark room in the absence of light in clear 1.5 ml Eppendorf micro-tubes followed by incubating the samples for 5 minutes on ice. The samples were then exposed for 3 minute to a 500 W halogen light source (Osram, T3Q clear halogen), which was 20 cm distant from the sample tubes while keeping the samples on ice.

Subsequently, the master mix was portioned out into PCR microfuge tubes, the serially diluted template DNA added to each sample, prior to a brief centrifugation step to collect all the reaction mixture on the bottom of the tube. The tubes were then placed into the Mastercycler and the amplification reactions started.

To determine if residual EMA had an adverse effect on the AmpliTaq® polymerase activity, a master mix was set up containing 3 μg ml⁻¹ of EMA but no added Taq polymerase. The master mix was further incubated and photolysed as described.

Subsequently, the master mix was aliquoted into PCR tubes and AmpliTaq® polymerase added in two-fold serial dilutions ranging from 1.25 to 0.0097 U. PCR reactions were run on each of the dilutions after adding 100 pg of A. flavithermus template DNA. A control master mix was similarly prepared, but without addition of EMA.

1. 7. Effect of EMA Concentrations Greater than 5 μg ml⁻¹ on PCR Efficiency

The effect of EMA in concentrations above 5 μg ml⁻¹ on the efficiency of PCR was investigated with an AmpliTaq® polymerase master mix containing 10 μg ml⁻¹ of the dye.

Following the protocol outlined in section 2.6, the PCR master mix was incubated in darkness for 5 minutes and illuminated for 3 minutes. Subsequent to light exposure either 200 or 20 pg μl⁻¹ of partially DNase I digested bovine DNA was added to the master mix, and the samples incubated in darkness for a further 5 minutes on ice in order to allow the restricted bovine DNA to react with any unbound EMA. The master mixes were then distributed and an aliquot of the decimal serially diluted template DNA added to the PCR tubes and amplification reaction initiated as described under 2.4.

1. 8. Agarose Gel-Electrophoresis, Imaging and Evaluation

Following PCR, the amplification products were electrophoresed through a 1.5% (w/v) agarose gel (SeaKem, LE Agarose) at 55 Volts (Power Pack Model 250; Life Technologies, GIBCO BRL) using a HORIZON 11-14 electrophoresis box (Life Technologies, GIBCO BRL).

Gels were stained with ethidium bromide (20 μg ml⁻¹) for 15 minutes and de-stained with distilled water for 20 minutes. The gels were then visualized and images captured with an Alphalmager™ (Alpha Innotech, San Leandro, Calif., USA).

Evaluation of the digitalized agarose-gels was performed on the computer images with the ImageJ 1.32j software package from Wayne Rasband (National Institute of Health, USA) available online at http://rsb.info.nih.gov/ij/.

The software was used to determine the relative amount of the amplification products as a measure of their pixel density (gray value). Subsequent to opening the images into ImageJ both background subtraction and the adjustment of the brightness and contrast of the images were performed with the default settings of the ImageJ. The banding of the gels was analysed using the “plot profile” option. The data plot value coordinates were copied and pasted into Microsoft Excel 2004 and the data plotted with the line chart option.

2. Results

2.1. Determination of the Optimal EMA Concentration for the Elimination of Exogenous DNA Contamination in PCR Master Mix.

The optimal amount of EMA for the removal of exogenous DNA from the PCR master mix containing AmpliTaq® polymerase was determined over the concentration range of 0.1, 1, 3, 5 and 10 μg ml⁻¹. Template DNA concentrations covering the range from 10 ng to 10 fg of A. flavithermus DNA were used to assess the sensitivity of the master mix to EMA incorporation. The results from these experiments are shown in FIGS. 1 to 5.

The negative controls for each of FIGS. 1 to 5, which contained no template DNA and had no EMA added (lane 10 in FIGS. 1 to 5) all produced one to two amplicons of around 300 bp. Thus indicating the presence of amplifiable exogenous DNA in the PCR master mix.

This was in contrast to the negative controls containing EMA but no template DNA (lane 9 in each of FIGS. 1 to 5) where the generation of a PCR amplicon depended on the amount of EMA used.

EMA concentrations below 1 μg ml⁻¹ (lane 9 in FIGS. 1 and 2) were unable to completely remove all the exogenous DNA present in the PCR master mix and both reactions produced an appropriate amplicon. The amount of PCR product generated—measured relative to the gray value in the pixel density profile—varied significantly between both samples, with the negative control sample treated with 1 μg ml⁻¹ EMA (FIG. 2, lane 9) producing a noticeably smaller quantity of PCR product than that treated with 0.1 ug ml⁻¹ EMA (FIG. 1, lane 9).

EMA concentrations of 3 μg ml⁻¹ and above successfully removed all exogenous DNA from the master mix and no PCR amplicon was observed for these samples (FIGS. 3 to 5, lanes 9).

EMA concentrations of 5 μg ml⁻¹ and above caused PCR failure for DNA template concentrations of 10 pg and below (FIGS. 4 and 5, lanes 5 to 8).

Thus, the optimal amount of EMA for the elimination of exogenous DNA in this master mix was 3 μg ml⁻¹ (FIG. 3), while at the same time showing no interference of amplification of template DNA at concentrations as low as 10 fg.

2.2. Effect of EMA Concentrations Greater than 5 μg m⁻¹ on PCR Efficiency

There are two possible interpretations for the failure of PCR reactions containing 10 pg to 10 fg of template DNA and treated with 5 and 10 μg ml⁻¹ EMA (FIG. 4 and 5, lanes 5 to 8).

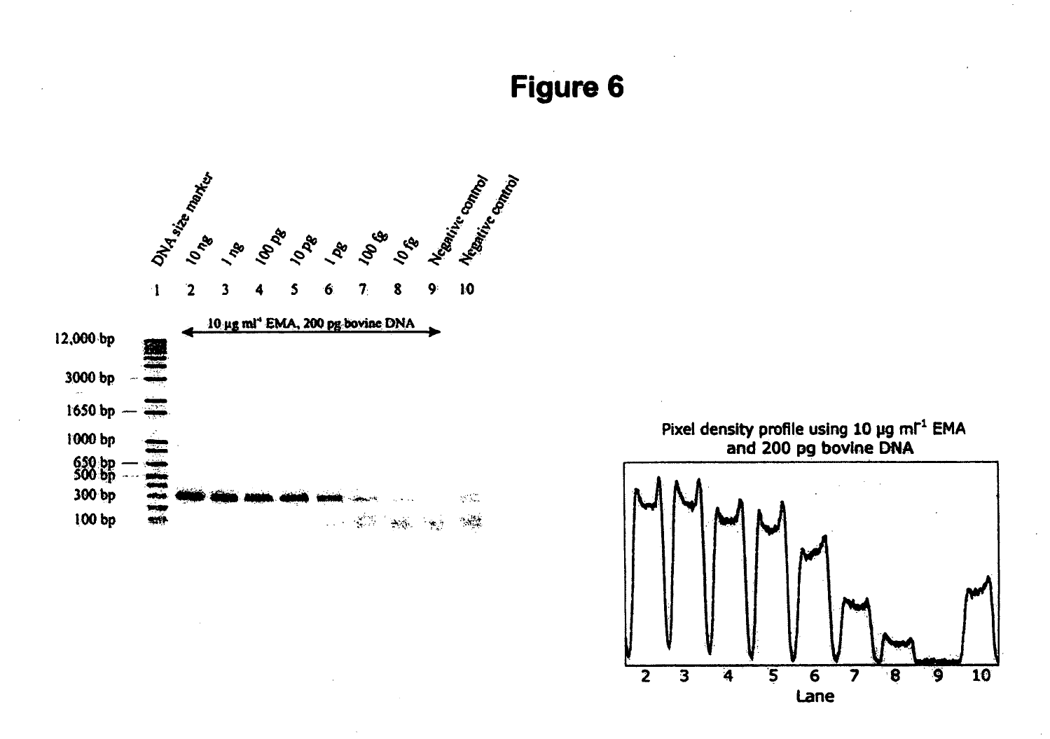

Firstly, that these higher concentrations of EMA partially inactivates the AmpliTaq® polymerase, and secondly, highly reactive but unbound nitrene due to excess EMA exists in the master mix, which subsequently complexes template DNA when it is added. The latter interpretation was tested by the addition of either 20 or 200 pg μl⁻¹ of DNase I digested bovine DNA to the PCR master mix prior to the addition of template DNA, but after EMA photolysis. This added DNA was not amplified by the primer set used, i.e. it would not compete with the template DNA. Addition of 20 pg μl⁻¹ of bovine DNA to the PCR master mix did not restore PCR performance at the lower concentrations of template DNA (data not shown). In contrast, the addition of 200 pg μI⁻¹ of bovine DNA to the master mix successfully restored the amplification reaction for the samples containing 10 pg to 10 fg of template DNA (FIG. 6).

These results indicate that the AmpliTaq® polymerase activity was not affected by the presence of relative high concentrations of EMA and that failure in amplification was likely due to template complexing by unreacted nitrene.

2.3. Effect of Residual EMA on Activity of AmpliTaq Polymerase and Template DNA

To further discount the effect of residual unreacted azide on the activity of AmpliTaq® polymerase and template DNA two further experiments were conducted.

In the first, the D-alanine racemace from Bacillus subtilis BS was amplified from 1 ng template DNA using PCR master mix preparations containing 0, 3 and 5 μg ml⁻¹ of EMA respectively.

The PCR products were subsequently sequenced, and their sequences compared to the known D-alanine racemase (1170 bp) of B. subtilis 168 by ClustalW alignment (as shown in Table 1). The results show that EMA at concentrations of 3 and 5 μg ml⁻¹, or any secondary products on photolysis, had no adverse effect on amplification and sequencing.

TABLE 1
Sequencing of D-alanine racemase of B. subtilis
BS (GenBank accession DQ291153)

| Sample | EMA ^{(a)} | Sequence | Score over full | Score over 1052 |
|---|---|---|---|---|
| No | [μg ml⁻¹] | length [bp] | sequence ^{(b)}[%] | bp ^{(b)}[%] |
| 1 | 3 | 1107 | 99 | 100 |
| 2 | 3 | 1109 | 99 | 100 |
| 3 | 5 | 1113 | 98 | 100 |
| 4 | 5 | 1113 | 98 | 100 |
| 5 | 0 | 1110 | 99 | 100 |

^{(a)} EMA added to the PCR master mix prior to photolysis and amplification.
^{(b)} Score absolute to the full length D-alanine racemase (1170 bp) of B. subtilis 168.

In the second experiment, aliquots of a two-fold dilution series of AmpliTaq® polymerase were added to a master mix treated with 3 μg ml⁻¹ EMA in order to determine if residual EMA had an adverse effect on the Taq polymerase activity.

The results derived from this experiment indicate that the end-point dilution between control (no added EMA) and EMA-treated master mixes was slightly different.

Amplification occurred in the control at an effective Taq polymerase concentration of 0.039 units per reaction, whereas the lowest concentration for the EMA treated sample was one dilution less. i.e. 0.079 units of Taq polymerase (data not shown). This equates to a loss of approximately 7% of added Taq polymerase activity and would not be noticeable under normal PCR operation.

2.4. Application to DNA Polymerases Other Than AmpliTaq Polymerase

The validation of the protocol from section 1.6 for the elimination of contaminating DNA in PCR master mix preparations was also undertaken on master mixes containing either the JumpStart Taq-polymerase or the Platinum Taq-Polymerase in the presence of 3 μg ml⁻¹ of EMA.

The results of both experiments are illustrated in FIGS. 7 and 8, and they reflect those from FIG. 3.

The untreated negative control samples from lanes 10 in FIG. 7 and 8 demonstrate the amplification of specific PCR amplicons, which substantiates that both DNA polymerases were the source of amplifiable DNA.

The density profiles indicate that quantitative difference in contaminating DNA were evident with the JumpStart Taq-polymerase introducing approximately 2 fg and the Platinum Taq-Polymerase approximately 40 fg of amplifiable DNA per reaction.

Crucially, treatment with 3 μg ml⁻¹ of EMA suppressed completely any amplification of the contaminating DNA in the negative control reactions (FIGS. 7 and 8 lane 9).

Moreover, both PCR master mixes were still able to amplify exogenously added template DNA from A. flavithermus in the range of 10 fg to 10 ng (FIGS. 7 and 8, lanes 2 to 8).

### REFERENCES

Aslanzahed, J. (1993). Application of hydroxylamine hydrochloride for post-PCR sterilization. Molecular and Cellular Probes 7(2), 145-150.

Bolton, P. H. and Kearns, D. R. (1978). Spectroscopic properties of ethidium monoazide: a fluorescent photoaffinity label for nucleic acid. Nucleic Acid Research 5(12), 4891-4903.

Böttger, E., C. (1990). Frequent contamination of Taq polymerase with DNA. Clinical Chemistry 36(6), 1258-1259.

Brosius, J., Dull, T. J., Sleeter, D. D., Noller, H. F. (1980). Gene organization and primary structure of a ribosomal RNA operon from Escherichia coli. Journal of Molecular Biology 148(2), 107-27.

Cantrell, C. E., Yielding, K. L., Pruitt, K. M. (1979). Efficiency of photolytic binding of ethidium monoazide to nucleic acids and synthetic polynucleotides. Molecular Pharmacology 15(2), 322-330.

Carroll, N. M., Adamson, P. and Okhravi, N. (1999). Elimination of bacterial DNA from Taq DNA polymerases by restriction endonuclease digestion. Journal of Clinical Microbiology 37(10), 3402-3404.

Corless, C. E., Guiver, M., Borrow, R., Edwards-Jones, V., Kaczmarski, E. B. and Fox. A. J. (2000). Contamination and sensitivity issues with a real-time universal 16S rRNA PCR. Journal of Clinical Microbiology 38(5), 1747-1752.

DeFililppes, F. M. (1991). Decontaminating the Polymerase Chain Reaction. Biotechniques 10(1), 26-30.

Deragon, J. M., Sinnett, D., Mitchell, G., Potier, M. and Labuda D. (1990). Use of gamma irradiation to eliminate DNA contamination for PCR. Nucleic Acids Research 18(20), 6149.

Fahle, G. A., Gill, V. J. and Fischer, S. H. (1999). Optimal activation of isopsoralen to prevent amplicon carryover. Journal of Clinical Microbiology 37(1), 261-262.

Freeze, E., Bautz, E. and Freese, E. B. (1961). The chemical and mutagenic specificity of hydroxylamine. Proceedings of the National Academy of Science of the USA 47, 845-855.

Frothingham. R., Blitchington, R. B., Lee, D. H., Greene, R. C., Wilson, K. H., (1992). UV absorption complicates PCR decontamination. Biotechniques 13, 208-210.

Garland, F., Graves, D., E., Yielding, L., W. and Cheung, H., C. (1980). Comparative studies of the binding of ethidium bromide and its photoreactive analogues to nucleic acids by fluorescence and rapid kinetics. Biochemistry 19(14), 3221-3226.

Goldenberger, D., Altwegg, M. (1995). Eubacterial PCR: contaminating DNA in primer preparations and its elimination by UV light. Journal Microbiology Methods 21, 27-32.

Goto, M., Ando, S., Hachisuka, Y. and Yoneyama, T. (2005). Contamination of diverse nifH and nifH-like DNA into commercial PCR primers. FEMS Microbiologic Letters 246(1), 33-88.

Graves, D. E., Watkins, C. L. and Yielding, L. W. (1981). Ethidium bromide and its photoreactive analogues: spectroscopic analysis of deoxyribonucleic acid binding properties. Biochemistry 20(7), 1887-1892.

Hardwick J. M., von Sprecken R. S., Yielding, K. L., Yielding, L. W. (1984). Ethidium binding sites on plasmid DNA determined by photoaffinity labeling. Journal of Biological Chemistry 1259(17), 11090-11097.

Heininger, A., Binder, M., Ellinger, A., Botzenhart, K., Unertl, K., and Dbring, G., (2003). DNase pretreatment of master mix reagents improves the validity of universal 16S rRNA gene PCR results. Journal of Clinical Microbiology 41(4), 1763-1765.

Hilali, F., Saulnier, P., Chachaty, E. and Andremont, A. (1997). Decontamination of polymerase chain reaction reagents for detection of low concentrations of 16S rRNA genes. Molecular Biotechnology 7(3), 207-16.

Hughes, M. S., Beck, L. A. and Skuce, R. A. (1994). Identification and elimination of DNA sequences in Taq DNA polymerase. Journal of Clinical Microbiology 32(8), 2007-2008.

Jinno, Y., Yoshiura, K. and Niikawa, N. (1990). Use of psoralen as extinguisher of contaminated DNA in PCR. Nucleic Acid Research 18(22), 6739.

Kitchin, P. A., Szotyori, A., Fromholc, C. and Almond N., 1990. Avoidance of false positive. Nature 344(6263), 201.

Maiwald, M., Ditton, H. J., Sonntag, H. G., von Knebel Doeberitz, M. (1994). Characterization of contaminating DNA in Taq-polymerase which occurs during amplification with a primer set for Legionella 5S ribosomal RNA. Molecular and Cellular Probes 8(1), 11-14.

Meier, A., Persing, D. H., Finken, M. and Böttger, E. C. (1993). Elimination of contaminating DNA within polymerase chain reaction reagents: implications for a general approach to detection of uncultured pathogens. Journal of Clinical Microbiology 31 (3), 646-652.

Millar, B. C., Xu, J. and Moore, J. E. (2002). Risk assessment models and contamination management: implications for broad-range ribosomal DNA PCR as a diagnostic tool in medical bacteriology. Journal of Clinical Microbiology 40(5), 1575-1580.

Mohammadi, T., Reesink, H. W., Vandenbroucke-Grauls, C. M. J. E. and Savelkoul, P. H. M. (2003). Optimization of real-time PCR assay for rapid and sensitive detection of eubacterial 16S ribosomal DNA in platelet concentrates. Journal of Clinical Microbiology 41(10), 4796-4798.

Newsome, T. L. B. J., Zou, N. and Lo, S. C., (2004) Presence of bacterial phage-like DNA sequences in commercial Taq DNA polymerase reagents. Journal of Clinical Microbiology, 42(5), 2264-2267.

Nogva, H. K., Dromtorp, S.M., Nissen, H. and Rudi, K. (2003) Ethidium monoazide for DNA-based differentiation of viable and dead bacteria by 5′-nuclease PCR. Biotechniques 34(4), 804-813.

Ou, C. Y., Moore, J. L. and Schochetman, G., (1991) Use of UV irradiation to reduce false positivity in polymerase chain reaction. Biotechniques 10(4), 442-444.

Prince, A. M. and Andrus, L. (1992). PCR: how to kill unwanted DNA. Biotechniques 12(3), 358-360.

Rand, K. H., and Houck, H. (1990). Taq polymerase contains bacterial DNA of unknown origin. Molecular and Cellular Probes 4, 445-450.

Regan, J. M., Oldenburg, P. S., Park, H.-D., Harrington, G., W. and Noguera, D. R. (2003).

Simultaneous determination of bacterial viability and identity in biofilms using ethidium monoazide and fluorescent in situ hybridization. Water Science & Technology 47(5), 123-128.

Riedy, M. C., Muirhead, K. A., Jensen, C. P. and Stewart, C. C. (1991). Use of a photolabeling technique to identify nonviable cells in fixed homologous or heterologous cell populations. Cytometry 12(2), 133-139.

Rochelle, P. A., Weightman, A. J. and Fry, J. C. (1992). DNase I treatment of Taq DNA polymerase for complete PCR decontamination. Biotechniques 13, 520.

Ronimus, R. S., Parker, L. E., Turner, N., Poudel, S., Ruckert, A. and Morgan, H. W. (2003). A RAPD-based comparison of thermophilic bacilli from milk powders. International Journal of Food Microbiology 85(1-2), 45-61.

Rudi, K., Moen, B., Dromtorp, S. M. and Holck, A. L. (2005). Use of ethidium monoazide and PCR in combination for quantification of viable and dead cells in complex samples. Applied and Environmental Microbiology 71(2), 1018-1024. Rueckert, A., Ronimus, R. S. and Morgan, H. W. (2005). Development of a rapid detection and enumeration method for thermophilic bacilli in milk powders. Journal of Microbiological Methods 60, 155-167.

Saksena, N. K., Dwyer, D. and Barre-Sinausi. F. (1991). A “sentinel” technique for monitoring viral aerosol contamination. The Journal of Infectious Diseases 164, 1021-1022.

Sambrook, J., Russel, D. W. (2001). Molecular cloning. A laboratory manual. Cold Spring Harbor Laboratory Press, Cold Spring Harbor, N.Y.

Sarkar, G., and Sommer, S. S. (1990). Shedding light on PCR contamination. Nature 343(6253), 27.

Sarkar, G. and Sommer, S. S. (1991). Parameters affecting susceptibility of PCR contamination to UV inactivation. Biotechniques 10(5), 590-594.

Sarkar, G. and Sommer, S. S. (1993). Removal of DNA contamination in polymerase chain reaction reagents by ultraviolet irradiation. Methods in Enzymology 1218, 381-388.

Schmidt, T. M., Pace, B. and Pace, N. R. (1991). Detection of DNA contamination in Taq polymerase. Biotechniques 11 (2), 176-177.

Sharma, J. K., Gopalkrishna, V., and Das, B. C., (1992). A simple method for elimination of unspecific amplifications in polymerase chain reaction. Nucleic Acids Research 20(22), 6117-6118.

Wilson, K. H., Blitchington, R. B., and Greene, R. C. (1990). Amplification of bacterial 16S ribosomal DNA with polymerase chain reaction. Journal of Clinical Microbiology 28(9), 1942-1946.

Yamamoto, K., Inoue, S. and Kawanishi, S. (1993). Site-specific DNA damage and 8-hydroxydeoxyguanosine formation by hydroxylamine and 4-hydroxyaminoquinoline 1-oxide in the presence of Cu(II): role of active oxygen species. Carcinogenesis 14(7), 1397-1401.

Aspects of the present invention have been described by way of example only and it should be appreciated that modifications and additions may be made thereto without departing from the scope thereof as defined in the appended claims.

1. A method of treating a solution prior to a DNA amplification reaction, including the steps of:
(a) taking a base solution, which is void of template DNA for an amplification reaction;
(b) mixing a binding agent which has at least one active form and at least one inactive form with the base solution;
(c) subjecting the mixture from (b) to an external stimulus which converts the binding agent from an inactive to active form, causing the binding agent to bind to any contaminating DNA in the base solution so that the contaminating DNA is unreactive and is not co-amplified when the solution is subsequently utilised in a DNA amplification reaction during which the template DNA is added; and
(d) removing the external stimulus thereby converting the binding agent from an active to an inactive form.

2. A method of preparing a solution for use in DNA amplification reactions, including the steps of:
(a) taking a base solution, which is void of template DNA for an amplification reaction;
(b) determining the amount of contaminating exogenous DNA in a base solution;
(c) determining the concentration of a binding agent which has at least one active form and at least one inactive form required to bind all the contaminating DNA;
(d) mixing the required concentration of binding agent which has at least one active form and at least one inactive form with the base solution; and
(e) subjecting the mixture from (d) to an external stimulus which converts the binding agent from an inactive and active form, and causing the binding agent to bind to any contaminating DNA in the base solution so that the contaminating DNA is unreactive and is not co-amplified when the solution is subsequently utilised in a DNA amplification reaction during which the template DNA is added.
(f) removing the external stimulus thereby converting the binding agent from an active to an inactive form.

3. The method as claimed in claim 1, wherein the base solution is a protein solution.

4. The method as claimed in claim 1, wherein the base solution is a DNA polymerase solution.

5. The method as claimed in claim 1, wherein the base solution is a Taq polymerase solution.

6. The method as claimed in claim 1, wherein the base solution is a PCR master mix.

7. The method as claimed in claim 1, wherein the base solution is an RNA preparation.

8. The method as claimed in claim 1, wherein the DNA amplification reaction is PCR.

9. The method as claimed in claim 1, wherein the stimulus is bright white light.

10. The method as claimed in claim 1, wherein the stimulus is UV light.

11. The method as claimed in claim 1, wherein the binding agent is ethidium monoazide.

12. The method as claimed in claim 11, wherein the concentration of ethidium monoazide is between 1 and 5 μg mL⁻¹.

13. The method as claimed in claim 11, wherein the concentration of ethidium monoazide is in the order of 3 μg mL⁻¹.

14. A solution for use in DNA amplification reactions, wherein the solution is produced via the method as claimed in claim 1.

15. A method of DNA amplification, including the steps of:
(a) preparing a solution using the method as claimed in claim 1; and,
(b) using the solution from a) in a DNA amplification reaction.

16. (canceled)

17. (canceled)

18. (canceled)


SEQUENCE LISTING (4 sequences)

SEQ ID NO: 1 - DNA, 19 bp, bacilli
aggaacaccagtggcgaag

SEQ ID NO: 2 - DNA, 21 bp, bacilli
ggatgtcaagacctggtaagg

SEQ ID NO: 3 - DNA, 26 bp, B.subtilis strain 168
agcacaaaacctttttacagagatac

SEQ ID NO: 4 - DNA, 29 bp, B.subtilis strain 168
ttaattgcttatatttacctgcaataaag